(19) United States
(12) Patent Application Publication
(10) Pub. No.: US 2020/0273584 A1
(43) Pub. Date: Aug. 27, 2020

(54) METHOD AND APPARATUS FOR MONITORING A SUBJECT

(71) Applicant: KONINKLIJKE PHILIPS N.V., EINDHOVEN (NL)

(72) Inventors: Alberto Giovanni BONOMI, Eindhoven (NL); Warner Rudolph Theophile TEN KATE, Waalre (NL); Gabriele PAPINI, Eindhoven (NL)

(21) Appl. No.: 16/066,229

(22) PCT Filed: Dec. 22, 2016
(86) PCT No.: PCT/EP2016/082323

§ 371 Date: Jun. 26, 2018

(30) Foreign Application Priority Data
Jan. 5, 2016 (EP) 16150118.4

(51) Int. Cl. G16H 50/50; G16H 40/67; G16H 50/20; G16H 20/60

(52) U.S. Cl. CPC G16H 50/50; G16H 20/60; G16H 50/20; G16H 40/67

(57) ABSTRACT

Method and apparatus for monitoring a subject There is provided a method of monitoring a subject, the method comprising obtaining measurements of a physiological characteristic of the subject over a period of time; obtaining information on a desired trend for the physiological characteristic of the subject; analysing the measurements of the physiological characteristic in a first time interval to determine a global trend having a global upper trend line for the physiological characteristic based on maxima in the measurements in the first time interval and a global lower trend line for the physiological characteristic based on minima in the measurements in the first time interval; analysing the measurements of the physiological characteristic in a second time interval to determine a local trend for the physiological characteristic, wherein the second time interval is shorter than the first time interval; determining whether feedback to the subject is required based on the global trend, the local trend and the desired trend; and if feedback is required, providing the feedback to the subject.

### TECHNICAL FIELD OF THE INVENTION

The invention relates to a method and apparatus for monitoring a subject and in particular relates to a method and apparatus for monitoring a physiological characteristic of a subject.

### BACKGROUND TO THE INVENTION

The maintenance of a particular body weight is difficult in modern society because of easy access to calorific food. When calorie intake exceeds calorie expenditure, body weight increases causing the person to be overweight or even obese. In addition, the lack of proper nutrition and weight loss can be a serious issue in the elderly population where biological signalling of hunger and satiety may be inadequate. Therefore the issue of maintaining a healthy body weight is a pressing societal challenge and an unmet consumer need.

Calorie intake tends to occur intermittently and rather regularly, and the daily routine can be characterized by food intake events such as breakfast, lunch, snacking and dinner. Similarly, opportunities to burn calories occur according to our daily habitual routine. Active commuting (e.g. walking, cycling, driving, etc.), physical exercise, occupational tasks, house chores and sedentary moments tend to be highly repetitive on a daily and weekly basis. With such repetitive behaviour maintaining a balance between calorie intake and calorie expenditure should be a feasible target for most healthy individuals. However, events leading to disturbances in the personal routine may cause an unbalanced energy budget and lead to rapid weight gain or weight loss. Events such as holidays, celebrations, social occurrences, travel, illness, stress periods and work deadlines may modify habits and calorie intake to an extent that individuals are unable to adapt to in order to achieve calorie/energy balance. In addition, reversing the effect of any weight gain can be particularly difficult requiring often long-term effort and dieting programs.

FIG. 1 illustrates the concept that sporadic events can modify the personal routine by encouraging unusual eating and/or activity behaviour, leading to abnormal calorie intake and an unbalanced energy budget which determines weight gain or loss. The top part of FIG. 1 represents the subject being in their normal personal routine (box 80) with eating and activity behaviour being normal for the subject. In this normal routine the subject has a normal calorie intake and has achieved an energy balance (box 82) and their body weight is stable (subject to an expected daily/weekly variability)—box 84. The lower part of FIG. 1 illustrates the effect of a non-routine event that results in modified eating and activity behaviour (box 90) for the subject that leads to an unusual level of calorie intake and thus disrupts the energy balance (box 92) leading to weight gain or loss (as appropriate)—box 94.

A subject's weight varies on a daily or weekly basis according to the subject's normal routine, and it is difficult to reliably determine the early occurrence of weight gain or weight loss from a small number of weight measurements. Subjects concerned about their weight may measure themselves on a daily basis. Typically, a measurement is taken at a regular time during the day, for example when going to bed or when waking up. The measurement is compared to some reference by the subject to decide whether he or she has lost weight or gained weight.

Systems are available that can be used to help encourage a subject to achieve a weight goal by providing the subject with warnings about their weight. For example for a subject that is intending to maintain their current weight, they should receive a warning that their weight is incrementing as early as possible. This is because when the warning is issued at an early stage, the amount of effort needed to correct the increment is also minimized.

EP 2363061 is an example of a system for monitoring and managing body weight and other physiological conditions which aims to achieve an optimum or preselected energy balance between calories consumed and energy expended by the user, and provide feedback to the user.

However, systems such as those described in EP 2363061 tend to provide the user with feedback and warnings on a frequent basis, for example as soon as there is a change in the user's weight (which can occur daily and/or regularly according to their personal routine), and it has been found that users become less responsive to these feedback and warnings, which ultimately means that the system is not successful in helping the user to achieve their goal.

Other background information can be found in US 2012/0313776 which describes a general health and wellness management method and apparatus for a wellness application using data from a data-capable band, and US 2008/0162352 describes a health maintenance system for health assessment, abnormality detection, health monitoring, health pattern and trend detection, health strategy development and health history archiving.

Therefore there is a need for an improved method and apparatus for monitoring a subject that overcomes this problem.

### SUMMARY OF THE INVENTION

According to a first aspect, there is provided a method of monitoring a subject, the method comprising obtaining measurements of a physiological characteristic of the subject over a period of time; obtaining information on a desired trend for the physiological characteristic of the subject; analysing the measurements of the physiological characteristic in a first time interval to determine a global trend having a global upper trend line for the physiological characteristic based on maxima in the measurements in the first time interval and a global lower trend line for the physiological characteristic based on minima in the measurements in the first time interval; analysing the measurements of the physiological characteristic in a second time interval to determine a local trend for the physiological characteristic, wherein the second time interval is shorter than the first time interval; determining whether feedback to the subject is required based on the global trend, the local trend and the desired trend; and if feedback is required, providing the feedback to the subject.

Thus, this method can be used to avoid feedback being provided to a subject as soon as the subject appears to start deviating from a desired trend (which can occur frequently for a physiological characteristic that naturally varies up and down on a daily basis), and provide feedback when the subject's long term behaviour (as indicated by the global trend) suggests that the deviation from the desired trend is more than just natural variation in the physiological characteristic. Thus, the method can reduce the amount of unnecessary warnings and alerts provided to the subject, which should improve the responsiveness of the subject to the warning or alert when it is issued.

In some embodiments, the desired trend comprises an indication of whether the physiological characteristic should be increased, decreased or maintained at a stable value.

In some embodiments, the step of analysing the measurements of the physiological characteristic in the first time interval comprises determining the global upper trend line as a line that connects two or more maxima in the measurements of the physiological characteristic in the first time interval, and determining the global lower trend line as a line that connects two or more minima in the measurements of the physiological characteristic in the first time interval. The two or more maxima can be a greater time apart than the second time interval and the two or more minima can be a greater time apart than the second time interval.

In some embodiments, the step of analysing the measurements of the physiological characteristic in the second time interval to determine the local trend for the physiological characteristic comprises determining the local trend as a moving average of the measurements of the physiological characteristic in the second time interval.

In some embodiments, the step of determining whether feedback to the subject is required comprises determining whether the global trend is consistent with the desired trend; and determining whether the local trend is between the global upper trend line and the global lower trend line.

In some embodiments, the step of determining whether feedback to the subject is required comprises: determining whether the global trend is consistent with the desired trend; if the global trend is consistent with the desired trend, determining whether the local trend is between the global upper trend line and the global lower trend line; determining that negative feedback to the subject is required if the local trend is not between the global upper trend line and the global lower trend line; if the global trend is not consistent with the desired trend, determining whether the local trend is consistent with the desired trend; determining that negative feedback to the subject is required if the local trend is not consistent with the desired trend; and otherwise, determining that no feedback to the subject is required. Thus, this embodiment provides the advantage that feedback can be provided if both the local trend and the global trend indicate that over the long term the goal for the physiological characteristic will not be met, or if the local trend exceeds the ‘normal’ boundaries provided by the global trend which can occur when there has been some significant recent change in the behaviour or circumstances of the subject that has affected the physiological characteristic. However, this embodiment recognises that negative feedback should not be provided if the difference in the global trend and desired trend has recently been corrected by the subject (by getting the local trend consistent with the desired trend), for example by making positive changes to their behaviour or lifestyle.

In some embodiments, the step of determining whether feedback to the subject is required comprises: determining whether the global trend is consistent with the desired trend; if the global trend is consistent with the desired trend, determining whether the local trend is between the global upper trend line and the global lower trend line; determining that negative feedback to the subject is required if the local trend is not between the global upper trend line and the global lower trend line; if the global trend is not consistent with the desired trend, determining whether the local trend is consistent with the desired trend; if the local trend is not consistent with the desired trend, determining whether the local trend is between the global upper trend line and the global lower trend line; determining that negative feedback to the subject is required if the local trend is not between the global upper trend line and the global lower trend line; and otherwise, determining that no feedback to the subject is required. Thus, this embodiment provides the advantage that feedback can be provided if both the local trend and the global trend indicate that over the long term the goal for the physiological characteristic will not be met and the physiological characteristic is now outside of the normal bounds for the subject, or if the local trend exceeds the ‘normal’ boundaries provided by the global trend, both of which can occur when there has been some significant recent change in the behaviour or circumstances of the subject that has affected the physiological characteristic. However, this embodiment also recognises that negative feedback should not be provided if the difference in the global trend and desired trend has recently been corrected by the subject (by getting the local trend consistent with the desired trend), for example by making positive changes to their behaviour or lifestyle.

In some embodiments, the method further comprises the steps of: obtaining information on the activities, events and/or behaviour of the subject over said period; and analysing the information on the activities, events and/or behaviour and the measurements of the physiological characteristic to determine associations between certain activities, events and/or behaviour of the subject and increases in the physiological characteristic, decreases in the physiological characteristic and/or stability in the physiological characteristic.

In some embodiments, if it is determined that feedback is required, the method further comprises the step of determining the feedback to be provided to the subject based on the desired trend and the determined associations. This embodiment provides the advantage that the feedback can indicate certain activities, events and/or behaviour that the subject could adopt or undertake in order to correct the anomalous situation that has led to the negative feedback.

In some embodiments, the method further comprises the steps of obtaining information on upcoming activities, events and/or behaviour for the subject in the second time interval; using the determined associations to analyse the upcoming activities, events and/or behaviour to predict whether the upcoming activities, events and/or behaviour will lead to an increase in the physiological characteristic, a decrease in the physiological characteristic and/or the physiological characteristic being stable.

In some embodiments, the step of analysing the measurements of the physiological characteristic in the second time interval to determine the local trend for the physiological characteristic comprises using the measurements of the physiological characteristic in the second time interval and the prediction based on upcoming activities, events and/or behaviour for the subject to determine the local trend. This embodiment provides the advantage that some prediction of the trend for the physiological characteristic can be made based on how certain upcoming activities, events and/or behaviour have affected the physiological characteristic of the subject in the past, and therefore feedback can be provided prior to those activities, events and/or behaviour that they may adversely affect the physiological characteristic.

According to a second aspect of the invention, there is provided a computer program product comprising a computer readable medium having computer readable code embodied therein, the computer readable code being configured such that, on execution by a suitable computer or processor, the computer or processor is caused to perform any of the methods described above.

According to a third aspect of the invention, there is provided an apparatus for monitoring a subject, the apparatus comprising a control unit and a user interface, wherein the control unit comprises a first obtaining module for obtaining measurements of a physiological characteristic of the subject over a period of time; a second obtaining module for obtaining information on a desired trend for the physiological characteristic of the subject; a first analysing module for analysing the measurements of the physiological characteristic in a first time interval to determine a global trend having a global upper trend line for the physiological characteristic based on maxima in the measurements in the first time interval and a global lower trend line for the physiological characteristic based on minima in the measurements in the first time interval; a second analysing module for analysing the measurements of the physiological characteristic in a second time interval to determine a local trend for the physiological characteristic, wherein the second time interval is shorter than the first time interval; a determining module for determining whether feedback to the subject is required based on the global trend, the local trend and the desired trend; and wherein the user interface is configured to provide the feedback to the subject if it is determined that feedback is required.

Thus, the apparatus can be used to avoid feedback being provided to a subject as soon as the subject appears to start deviating from a desired trend (which can occur frequently for a physiological characteristic that naturally varies up and down on a daily basis), and provide feedback when the subject's long term behaviour (as indicated by the global trend) suggests that the deviation from the desired trend is more than just natural variation in the physiological characteristic. Thus, the apparatus should provide less warnings and alerts to the subject, which should improve the responsiveness of the subject to a warning or alert when it is issued.

In some embodiments, the desired trend comprises an indication of whether the physiological characteristic should be increased, decreased or maintained at a stable value.

In some embodiments, the first analysing module is configured to analyse the measurements of the physiological characteristic in the first time interval by determining the global upper trend line as a line that connects two or more maxima in the measurements of the physiological characteristic in the first time interval, and determining the global lower trend line as a line that connects two or more minima in the measurements of the physiological characteristic in the first time interval. The two or more maxima can be a greater time apart than the second time interval and the two or more minima can be a greater time apart than the second time interval.

In some embodiments, the second analysing module is configured to analyse the measurements of the physiological characteristic in the second time interval to determine the local trend for the physiological characteristic by determining the local trend as a moving average of the measurements of the physiological characteristic in the second time interval.

In some embodiments, the determining module is configured to determine whether feedback to the subject is required by determining whether the global trend is consistent with the desired trend; and determining whether the local trend is between the global upper trend line and the global lower trend line.

In some embodiments, the determining module is configured to determine whether feedback to the subject is required by: determining whether the global trend is consistent with the desired trend; if the global trend is consistent with the desired trend, determining whether the local trend is between the global upper trend line and the global lower trend line; determining that negative feedback to the subject is required if the local trend is not between the global upper trend line and the global lower trend line; if the global trend is not consistent with the desired trend, determining whether the local trend is consistent with the desired trend; determining that negative feedback to the subject is required if the local trend is not consistent with the desired trend; and otherwise, determining that no feedback to the subject is required. Thus, this embodiment provides the advantage that feedback can be provided if both the local trend and the global trend indicate that over the long term the goal for the physiological characteristic will not be met, or if the local trend exceeds the ‘normal’ boundaries provided by the global trend which can occur when there has been some significant recent change in the behaviour or circumstances of the subject that has affected the physiological characteristic. However, this embodiment recognises that negative feedback should not be provided if the difference in the global trend and desired trend has recently been corrected by the subject (by getting the local trend consistent with the desired trend), for example by making positive changes to their behaviour or lifestyle.

In some embodiments, the determining module is configured to determine whether feedback to the subject is required by determining whether the global trend is consistent with the desired trend; if the global trend is consistent with the desired trend, determining whether the local trend is between the global upper trend line and the global lower trend line; determining that negative feedback to the subject is required if the local trend is not between the global upper trend line and the global lower trend line; if the global trend is not consistent with the desired trend, determining whether the local trend is consistent with the desired trend; if the local trend is not consistent with the desired trend, determining whether the local trend is between the global upper trend line and the global lower trend line; determining that negative feedback to the subject is required if the local trend is not between the global upper trend line and the global lower trend line; and otherwise, determining that no feedback to the subject is required. Thus, this embodiment provides the advantage that feedback can be provided if both the local trend and the global trend indicate that over the long term the goal for the physiological characteristic will not be met and the physiological characteristic is now outside of the normal bounds for the subject, or if the local trend exceeds the ‘normal’ boundaries provided by the global trend, both of which can occur when there has been some significant recent change in the behaviour or circumstances of the subject that has affected the physiological characteristic. However, this embodiment also recognises that negative feedback should not be provided if the difference in the global trend and desired trend has recently been corrected by the subject (by getting the local trend consistent with the desired trend), for example by making positive changes to their behaviour or lifestyle.

In some embodiments, an obtaining module is further for obtaining information on the activities, events and/or behaviour of the subject over said period; and an analysing module is further for analysing the information on the activities, events and/or behaviour and the measurements of the physiological characteristic to determine associations between certain activities, events and/or behaviour of the subject and increases in the physiological characteristic, decreases in the physiological characteristic and/or stability in the physiological characteristic.

In some embodiments, the determining module is for determining the feedback to be provided to the subject based on the desired trend and the determined associations if it is determined that feedback is required. This embodiment provides the advantage that the feedback can indicate certain activities, events and/or behaviour that the subject could adopt or undertake in order to correct the anomalous situation that has led to the negative feedback.

In some embodiments, an obtaining module is further for obtaining information on upcoming activities, events and/or behaviour for the subject in the second time interval; an analysing module is further for using the determined associations to analyse the upcoming activities, events and/or behaviour to predict whether the upcoming activities, events and/or behaviour will lead to an increase in the physiological characteristic, a decrease in the physiological characteristic and/or the physiological characteristic being stable.

In some embodiments, the second analysing module is configured to analyse the measurements of the physiological characteristic in the second time interval to determine the local trend for the physiological characteristic by using the measurements of the physiological characteristic in the second time interval and the prediction based on upcoming activities, events and/or behaviour for the subject to determine the local trend. This embodiment provides the advantage that some prediction of the trend for the physiological characteristic can be made based on how certain upcoming activities, events and/or behaviour have affected the physiological characteristic of the subject in the past, and therefore feedback can be provided prior to those activities, events and/or behaviour that they may adversely affect the physiological characteristic.

According to a fourth aspect of the invention, there is provided an apparatus for monitoring a subject, the apparatus comprising a control unit and a user interface, wherein the control unit is configured to obtain measurements of a physiological characteristic of the subject over a period of time; obtain information on a desired trend for the physiological characteristic of the subject; analyse the measurements of the physiological characteristic in a first time interval to determine a global trend having a global upper trend line for the physiological characteristic based on maxima in the measurements in the first time interval and a global lower trend line for the physiological characteristic based on minima in the measurements in the first time interval; analyse the measurements of the physiological characteristic in a second time interval to determine a local trend for the physiological characteristic, wherein the second time interval is shorter than the first time interval; determine whether feedback to the subject is required based on the global trend, the local trend and the desired trend; and wherein the user interface is configured to provide the feedback to the subject if it is determined that feedback is required.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 illustrates the effect of sporadic events on the weight of a subject;

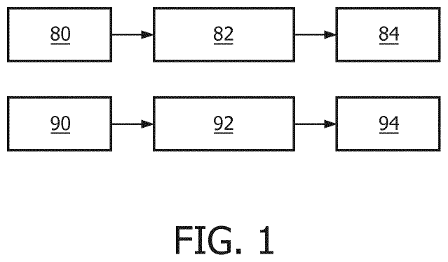

FIG. 2 is a graph illustrating changes in body weight during and following a holiday period;

FIG. 3 is a graph illustrating changes in body weight during and following a holiday period;

FIG. 4 is graph illustrating the analysis of stock price fluctuations;

FIG. 5 is a block diagram of an apparatus according to an embodiment;

FIG. 6 is a flow chart illustrating a method of monitoring a subject according to an embodiment of the invention;

FIG. 7 is a flow chart illustrating an exemplary method for implementing step 109 in FIG. 6;

FIG. 8 is a flow chart illustrating additional exemplary steps in the method according to the invention; and

FIG. 9 illustrates the clustering of user situations according to information on occupational, leisure-time and activity features.

### DETAILED DESCRIPTION OF THE PREFERRED EMBODIMENTS

Although some of the following description of the invention is presented in terms of a method and apparatus for monitoring the weight of a subject (whether for coaching the subject to lose weight, to gain weight or to maintain their weight), it will be appreciated that the method and apparatus can readily be applied to monitoring other types of physiological characteristic of a subject that fluctuate on a short term basis and that can vary more significantly over a relatively long timescale (e.g. days, weeks, months, etc.) and for which it may be useful to provide feedback to the subject to enable the subject to take corrective/preventative action to achieve a desired aim for that characteristic (e.g. increase, decrease, stable). For example, suitable physiological characteristics include blood pressure, resting heart rate, fat free/lean mass, fat mass, body fluid levels, waist circumference, waist-to-hip ratio, blood cholesterol level, blood glucose level, breathing rate, coughing frequency, walking speed, walking regularity, (foot)step frequency, etc.

As noted above, a subject's weight varies on a daily or weekly basis according to the subject's normal routine. Body weight fluctuates between measurements since the measurement conditions are not strictly identical. The body has natural fluctuations in weight that are not directly linked to excessive or insufficient food intake. Body weight may vary from day to day because of differences in hydration and fluid retention, which are influenced by sweating, fever, hormonal influences or physical exertion. Changes in body weight due to changes in fluid content may be uninformative as they are unrelated to accumulation or reduction of body fat, yet they cause consistent variations on a day-to-day basis. Also, both food intake and activity levels vary from day to day, leading to another cause of natural fluctuation. These natural fluctuations make it difficult to detect (from the weight measurement) the onset of weight increase due to (systematic) excessive food intake or reduced activity.

Clearly it is desirable to avoid providing a subject with a warning or coaching message when their weight is fluctuating normally. Likewise, when a subject's weight is intentionally increasing or decreasing, there will be fluctuations in the weight measurements as the weight increases or decreases, and it is again desirable to avoid providing a subject with a warning or coaching message while their weight is still generally heading in the right direction. Of course, as noted above, in order to minimise the effort required to keep or get the subject's weight on track (whether increasing, decreasing or keeping the weight stable), it is desirable to provide the subject with a warning or coaching message as early as possible. Thus, the invention aims to identify situations in which the subject's weight (or more generally any physiological characteristic of the subject) is deviating from a target or goal and in which feedback should be provided to the subject.

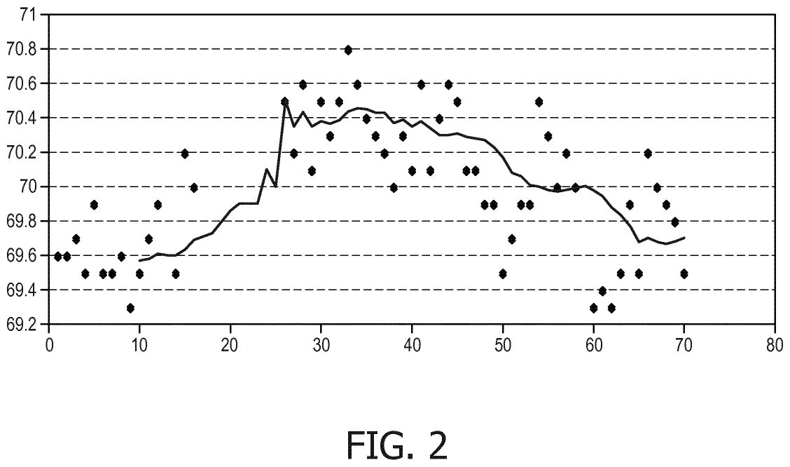

The graph in FIG. 2 shows changes in body weight in kilograms (kg) along the y-axis recorded for a subject during a holiday period of ten days from day 15 to day 25 (with time in days shown along the x-axis), and shows that the baseline weight value of 69.6 kg was only achieved again after around forty days following the event that caused the change in the daily routine. Each measurement point in FIG. 2 represents a weight measurement and the line is a moving average representation of the weight measurements. Thus, it can be seen from this graph that an early warning or coaching message around day 20 may have led the subject to change their behaviour before the end of the holiday (e.g. by reducing food intake), thereby reducing the total weight gain, and thereby reducing the time it took to return to their normal weight.

It has been found that identifying and anticipating the occurrence of events causing undesired variations in the personal routine (such as the holiday shown in FIG. 2) may be extremely useful in generating effective coaching (feedback) strategies to enable a subject to avoid unhealthy weight gain or loss. Thus, specific embodiments of the invention described below examine the subject's upcoming schedule for events or activities that may change the subject's calorie intake or activity level (based on previous occurrences of those events) and the apparatus aims to help subjects anticipate the effect of those events by preventing or adapting calorie intake to avoid an expected weight gain/loss.

Thus, in certain embodiments, the invention generates feedback for the subject in the form of coaching messages to guide the subject in the process of modifying food and calorie intake in those situations in which eating behaviour is expected to deviate from the personal routine. Whether any deviation from the routine is undesired depends on an observed or expected change in body weight beyond what is considered a normal range. The normal fluctuation range in body weight and the personal routine of the subject can be established by daily and/or weekly monitoring of body weight, and the personal schedule, respectively, for example as provided by connected devices (weight scales, computer software, mobile devices, and wearables). Coaching messages can be generated to warn users of a potential risk of excessive weight gain or loss when certain undesired events (e.g. social or behavioural circumstances) occur as suggested by the personal schedule.

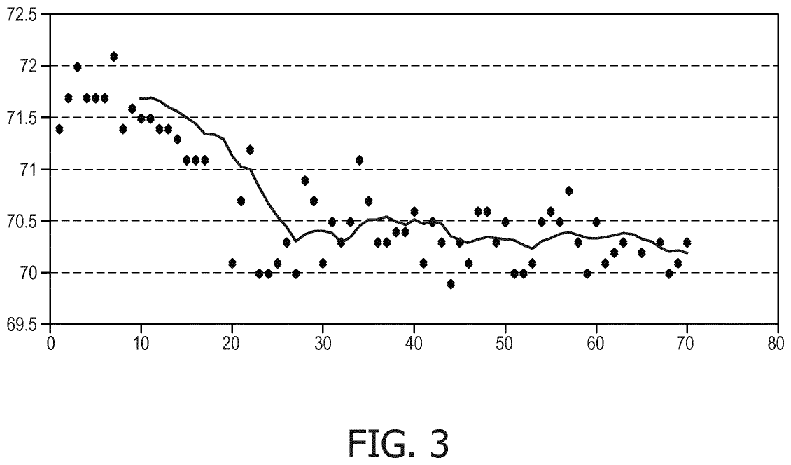

The graph in FIG. 3 shows changes in body weight recorded for a subject during an illness with a two-day fever from day 17 until day 20 (as with FIG. 2, time in days is shown on the x-axis and weight in kgs is shown on the y-axis). It can be seen that the illness led to a loss in weight until around day 26, and thereafter the weight was generally stable. It seems therefore that the routine of the subject after the illness (including activity levels and food intake) was sufficient for the subject to maintain a stable weight.

In some embodiments, similar to those described above with reference to FIG. 2, the apparatus can be arranged to monitor the behaviour (e.g. events, activities and/or food intake) of the subject to identify types of behaviour that lead to certain outcomes (e.g. weight gain, weight loss, weight stability), and then the apparatus can be arranged to recommend certain behaviour when a weight goal is set. For example, if it is desired for the subject to maintain a stable weight, the apparatus may recommend the behaviour (e.g. activity levels, food intake) associated with the period following the illness shown in FIG. 3 in which the subject's weight was stable.

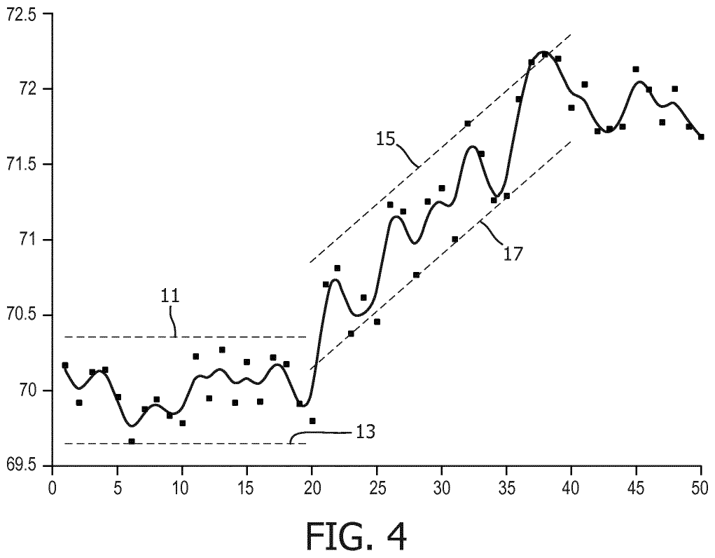

As described below, the invention enables the early and reliable detection of a deviation of a physiological characteristic from a desired outcome (e.g. increase, decrease or stability), even in the presence of daily fluctuations in the physiological characteristic. In particular, the invention analyses a time series of measurements of a physiological characteristic using techniques similar to those used to evaluate price movements of financial assets, such as shares. The graph in FIG. 4 illustrates the fluctuation in measured weight (measured in kgs and represented on the y-axis) over a period of 50 days (represented on the x-axis) and the use of trend lines to indicate the trend of the weight over a certain time interval. The points in the graph are the weight measurements and the solid line a form of average of the measured weight values.

For example, it can be seen that the weight is generally constant up to around day 20, and then the weight generally increases from day 20 to around day 40, where the weight then generally remains constant. Two trend lines 11, 13 are shown that indicate the generally constant weight up to day 20 (with trend line 11 being an upper trend line for the weight measurement up to day 20 and trend line 13 being a lower trend line for the weight measurement up to day 20). As another example, trend lines 15 and 17 are shown which bound the gradual increase in the weight measurement (with trend line 15 being an upper trend line for the weight measurement from day 20 to around day 40 and trend line 17 being a lower trend line for the weight measurement from day 20 to around day 40).

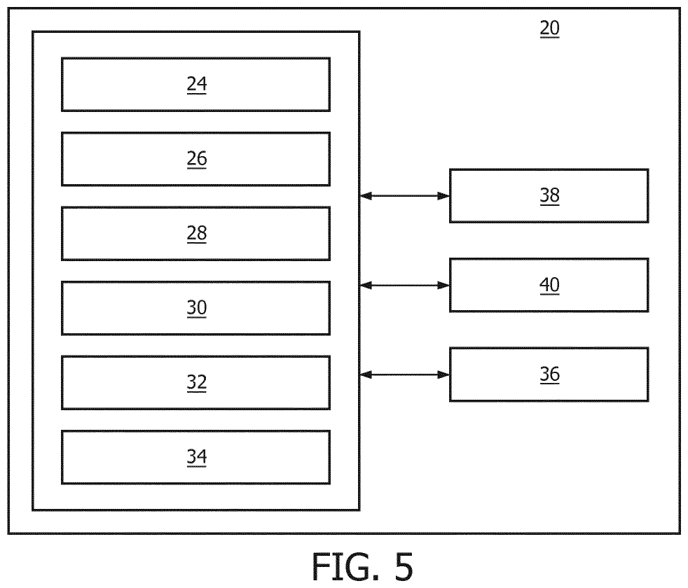

A block diagram of an apparatus 20 according to an embodiment of the invention is shown in FIG. 5. The apparatus 20 comprises a control unit 22 that controls the operation of the apparatus 20 and that can implement the monitoring method. Briefly, the control unit 22 is configured to process a time series of measurements of a physiological characteristic for a subject to determine whether feedback should be provided to the subject (and if so, what form that feedback should take). The control unit 22 can comprise one or more processors, processing units, multi-core processors or modules that are configured or programmed to control the apparatus 20 to monitor the subject as described below.

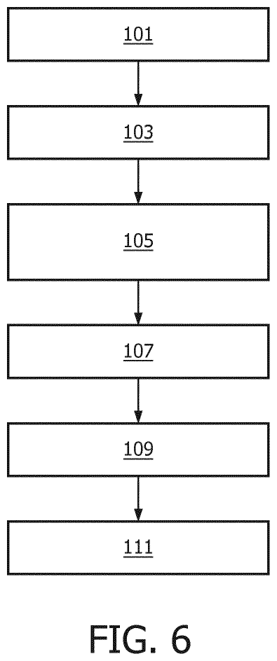

In particular implementations, the control unit 22 can comprise a plurality of software and/or hardware modules that are each configured to perform, or are for performing, individual steps in the monitoring method according to embodiments of the invention. As such, the control unit 22 (or more generally the apparatus 20) can comprise a First Obtaining module 24, Second Obtaining module 26, First Analysing module 28, Second Analysing module 30, Determining module 32 and Feedback module 34 that can each implement the functions required for performing steps 101-111 of FIG. 6 respectively, and which are described in more detail below.

In the illustrated embodiment, the apparatus 20 comprises a physiological characteristic sensor 36 that is for measuring one or more physiological characteristics of a subject. For example, in preferred embodiments where the apparatus 20 is for measuring weight, the sensor 36 can be a sensor, e.g. electronic weighing scales, for measuring the weight of a subject. In alternative embodiments, the sensor 36 can be a sensor for measuring blood pressure, heart rate, body composition (e.g. any one or more of fat free mass, fat mass, fluid levels), etc. as appropriate for the physiological characteristic that is to be monitored. In some embodiments weight and body composition can be measured (for example using weighing scales with a bioimpedance sensor) and the information combined to determine what might be causing weight to change. For example, it may be possible to effectively monitor a subject that is intending to increase their muscle mass and decrease their fat mass (but generally maintain a stable weight).

The sensor 36 can be part of the apparatus 20 or separate from the apparatus 20. In the embodiment of FIG. 5, the control unit 22 and the sensor 36 are shown as being part of the same device (e.g. within the same housing). However, it will be appreciated that the sensor 36 and control unit 22 can be provided in separate housings or devices, and they can be provided with appropriate circuitry or components to enable the measurement signal to be sent from the sensor 36 to the control unit 22.

In some embodiments the control unit 22 can receive a measurement directly from the sensor 36 and the control unit 22 can process this measurement along with previously-received measurements in order to determine whether feedback needs to be provided to the subject. In other embodiments (including embodiments where the sensor 36 is separate from the apparatus 20), a signal from a or the sensor 36 can be stored in a memory unit 38 and the control unit 22 can retrieve and analyse the previously-obtained sensor measurements from the memory unit 38 when it is to be determined whether feedback is to be provided to the subject.

The memory unit 38 can be used for storing program code that can be executed by the control unit 22 to perform the method described herein. The memory unit 38 can also be used to store signals and measurements made or obtained by the or a sensor 36 during operation.

In some embodiments the control unit 22 may be part of a smart phone or other general purpose computing device that can be connected to or otherwise receive a measurement signal from a sensor 36, but in other embodiments the apparatus 20 can be an apparatus that is dedicated to the purpose of monitoring a subject. In embodiments where the control unit 22 is part of a smart phone or other general purpose computing device, then depending on the physiological characteristic to be monitored, the sensor 36 could be a sensor that is integrated into the smart phone, or a sensor that is separate to the smart phone and that can provide sensor signals/measurements to the smart phone/computing device for processing and analysis (for example via a wired or wireless connection).

It will be appreciated that in some embodiments the apparatus 20 can make use of multiple sensors 36 (of the same or different types) to monitor the subject that can each be processed by control unit 22 to improve the reliability of the monitoring of the subject.

The apparatus 20 also comprises at least one user interface component 40 that is for use in providing the subject with feedback regarding their physiological characteristic.

For example the feedback can comprise an indication that the physiological characteristic is increasing/decreasing/stable or a target value for the physiological characteristic has been reached. The feedback can also or alternatively comprise feedback in the form of a warning that their current and/or predicted behaviour is causing or will cause an undesirable outcome for the physiological characteristic. The feedback can also or alternatively comprise coaching messages that inform the subject about ways in which they can change their action(s) in order to avoid an undesirable outcome for the physiological characteristic.

The user interface component 40 can comprise any component that is suitable for providing feedback or other information to the subject, and can be, for example, any one or more of a display screen or other visual indicator, a speaker, one or more lights, and a component for providing tactile feedback (e.g. a vibration function).

In addition, in some embodiments the user interface component 40 is or comprises some means that enables the subject or another user of the apparatus 20 to interact with and/or control the apparatus 20. For example, the user interface component 40 could comprise a switch, a button or other control means for activating and deactivating the apparatus 20 and/or monitoring process.

It will be appreciated that FIG. 5 only shows the components required to illustrate this aspect of the invention, and in a practical implementation the apparatus 20 will comprise additional components to those shown. For example, the apparatus 20 may comprise a battery or other power supply for powering the apparatus 20 or means for connecting the apparatus 20 to a mains power supply, and/or a communication module for enabling the measurements of the physiological characteristic of the subject to be communicated to a base unit for the apparatus 20 or a remote computer.

The flow chart in FIG. 6 illustrates a method of monitoring a subject according to an embodiment. This method can be implemented by a computer or by a control unit 20 as described above.

In a first step, step 101, which can be performed or implemented by the first obtaining module 24, measurements of a physiological characteristic of the subject over a period of time are obtained. The period of time is typically or preferably a number of days, weeks or months. Any number of measurements can be obtained over that time period, and they can be obtained multiple times per day, once per day, every other day, etc. This step can comprise obtaining one measurement at a time using sensor 36 (in which case step 101 can be repeated a plurality of times), or retrieving a previously-collected set of measurements of the physiological characteristic from a memory (e.g. from memory unit 38) or other form of data storage.

In a second step, step 103, which can be performed or implemented by the second obtaining module 26, information on a desired trend for the physiological characteristic of the subject is obtained. The information on a desired trend is information indicating how the subject (and/or perhaps a care provider) would like the physiological characteristic to change or evolve over time. For example, the information on a desired trend can indicate that it is desired or intended for the physiological characteristic to increase over time. Alternatively, the information on a desired trend can indicate that the physiological characteristic should decrease over time or remain stable (e.g. remain at a particular value or within a defined range of values). In some embodiments, the information on a desired trend can also indicate a target value for the physiological characteristic.

In embodiments where the physiological characteristic being monitored is weight, the information on a desired trend can indicate that the subject is to lose weight, gain weight or maintain a stable weight.

Step 103 can comprise receiving an input from the subject or another user of the apparatus 20 indicating the information on a desired trend, or retrieving the information on a desired trend from a memory (e.g. from memory unit 38) or other form of data storage.

In step 105, which can be performed or implemented by the first analysing module 28, the measurements of the physiological characteristic are analysed to determine a global trend for the physiological characteristic. This global trend is determined for measurements of the physiological characteristic in a first time interval. The first time interval can be a time interval that includes all of the measurements of the physiological characteristic or a subset of the measurements.

In the following explanation of step 105, reference is made to determining the global trend based on maxima and minima in the measurements of the physiological characteristic. It will be appreciated that maxima and minima may refer to actual maximum and minimum measurements, or to a group of neighbouring measurements that form a maxima or minima in a measurement signal. For example, the value of the maxima can be the average of a certain number of measurements (e.g. 3) that form a peak. A minima can be determined in a similar way. This understanding of maxima and minima is similar to the notion/concept of a ‘centre point’, which is a value that can be understood to represent a number in the middle (e.g. a mean, median or first mode) of a range of values. The ‘maxima’ can represent a number in the upper region of the range of values (without necessarily being the absolute maximum value). Minima can be understood in a similar way.

Generally the first time interval spans larger time periods, for example time periods that have a duration of a week or a month. In other embodiments it can be adjusted, or is parameterised, according to the time of the year (e.g. the season or month). For example, the time span itself has the duration of a week, but the value of its threshold is adapted to the current time of the year. In that sense the threshold is determined at the scale of a year.

The global trend can be determined by considering the envelope of the measurement data. The envelope can be found in one of several ways. One technique is based on Empirical Mode Decomposition (EMD) analysis: the local maxima and minima of the physiological characteristic measurements are determined and two lines connecting respectively the maxima and minima are computed. These two lines are the trend lines. The local maxima and minima are defined as the maximum (respectively minimum) at each moment over a time window of typically e.g. one week or one month. The window extends back in time for, e.g., half a week or half a month, and extends forward for, e.g., another half a week or half a month. It will be appreciated that defining the local maxima and minima in this way implies some latency to keep the system causal, as is known in the art. At the next measurement time instant the window shifts by the same amount (i.e. by the time between physiological characteristic measurements), and the maximum and minimum is again determined. It will be appreciated that these values can be the same, since the measurement providing the maximum or minimum may still be in the slightly shifted window.

Another technique that can be used to determine the envelope comprises computing the analytical signal, which, as known in the art, is obtained by expanding the physiological characteristic measurement signal with its Hilbert transform. The signal is written in analytical form as a complex signal: A(t). exp(j.φ(t)), where ϕ is the phase, and A is the envelope.

Another, more preferred, technique for determining the envelope uses a scheme like AM-demodulation. In AM (amplitude modulation) demodulation, the physiological characteristic measurement signal is “rectified” and subsequently low-pass filtered to extract the envelope (i.e. Amplitude modulation). In AM, the modulation is symmetrical (maxima and minima change in the same manner but with the opposite sign). In the present case, since an upper and lower envelope (upper and lower trend line) is desired, the scheme is modified as follows. The following explanation relates to determining the maxima (i.e. the upper trend line), and the minima (i.e. the lower trend line) can be determined in a similar way (with obvious modifications for signs (polarity)). Thus, for a new input sample (next physiological characteristic measurement), it is tested whether the measurement is larger than the current output sample. If so, the output sample is set to the value of that input sample. If not, the output sample is set to a fraction of its previous value, for example 0.95 times the previous value. In this way, the series of output samples will follow the envelope of maxima. The fraction parameter (e.g. 0.95) can be chosen according to the measurement rate (i.e. sampling rate) and the first time interval. Say, for example, the measurements of the physiological characteristic are large after a weekend, and there are four measurements per day. After a week has passed (if that is the time interval), there are 7*4=28 measurements, and the output signal has dropped to (0.95)̂28. This might be too low, and an attenuation of, e.g., 0.99 might be preferred. Also, an additional smoothing (low pass filter) might be applied to the obtained output signal.

The global trend determined in step 105 indicates the long term trend (long-range envelope) or fluctuations in the physiological characteristic over the first time interval.

Thus, as noted above, in preferred embodiments, the global trend comprises a global upper trend line for the physiological characteristic and a global lower trend line (which can represent the bounds of the envelope of the measurements). The global upper trend line can be based on maxima in the measurements of the physiological characteristic in the first time interval, and the global lower trend line can be based on minima in the measurements in the first time interval. In particular the global upper trend line can be a line that connects or relates to two or more maxima in the measurements, and the global lower trend line can be a line that connects or relates to two or more minima in the measurements. In the example shown in FIG. 4, trend line 11 corresponds to an upper global trend line that is based on peaks (although in this example the trend line does not touch any of the peaks), and trend line 13 corresponds to a lower global trend line that is based on the minima on day 6 (and it will be appreciated that in this example the first time interval would not include all of the data in FIG. 4, but a subset of the data, for example a 20 day period).

Thus, as noted above, in some embodiments, trend lines in measurements of a physiological characteristic are found by connecting respectively the local maxima and minima in the first time interval, e.g. corresponding to the previous week or month. These extremes can be found, for example by an algorithm in which clusters of large (or small in the case of the minima) values are identified, and the maxima (minima) in each cluster is determined. It will be noted above that the maxima or minima can correspond to an average of the values in each cluster rather than the absolute maximum and minimum values. Straight trend lines can be viewed as the first mode in an Empirical Mode Decomposition (EMD). In some embodiments, instead of determining a first order (straight) trend line, higher orders (polynomial or higher complexity) trend lines can be determined. It will be appreciated that horizontal trend lines can effectively be considered to define global upper and lower threshold values for the physiological characteristic.

In some embodiments, instead of identifying the maxima and minima, the center points of the measurements can be used (like the midpoint in EMD), to which an offset is added to determine the global upper trend line and respectively subtracted to arrive at the global lower trend line. In some embodiments, the offset can be estimated from the variance, for example as twice the standard deviation. In contrast to techniques for change detection, however, the trend lines are still adapted with respect to the maximum (and minimum) values, so that the trend line is close or even touching these maximum (and minimum) values.

Generally, however, it will be appreciated from the above that the upper and lower trend lines can be understood as representing the envelope of the measurements of the physiological characteristic that has been smoothed (e.g. low-pass filtered) over a long time scale.

In some cases the upper and/or lower trend line may be horizontal, indicating that the upper and/or lower trend for the physiological characteristic is constant (stable). Trend lines 11 and 13 in FIG. 4 are examples of constant trend lines. These constant trend lines can also be presented as global upper and lower threshold values since they each correspond to a single value for the physiological characteristic. In other cases the upper and/or lower trend line may be sloped (i.e. not horizontal), indicating that the upper and/or lower trend for the physiological characteristic is an increase or a decrease.

In step 107, which can be performed or implemented by the first analysing module 30, the measurements of the physiological characteristic in a second time interval are analysed to determine a local trend for the physiological characteristic. The second time interval is shorter than the first time interval, preferably substantially shorter. For example, whereas the first time interval can be several weeks or months, the second time interval will be several days or a week.

The second time interval is smaller than the first time interval. A typical length of the second time interval can be a few days, for example 10 days or a week (with the first interval being a month or more). As can be seen from FIGS. 2, 3 and 4, a window corresponding to the second time interval generally covers a range of weight values of about 0.5 kg, whereas a window corresponding to the first time interval has trend lines covering weight values of about 1.5 kg to 2 kg apart.

In some embodiments the local trend can be determined as a running mean or moving average of the measurements of the physiological characteristic in the second time interval. A time window based on the second time interval is used to characterise the bandwidth order/size of the filter/smoother, while the averaging is a typical implementation to realise smoothing, also parameterised by a time window. Any form of low-pass filtering can be used to determine the local trend. The filter might also be non-linear, such as a median filter, or any other form of ranked filter. For example, the filter could compute the maximum and minimum over the second time interval/window and take the average (this type of filter is known as a ‘midpoint’ filter). In other embodiments the local trend can be determined using local regression techniques.

In some embodiments, the second time interval is such that it includes current and recent measurements of the physiological characteristic, in which case the local trend is calculated from current and recent measurements of the physiological characteristic. In alternative embodiments, the second time interval can be such that it not only includes current and recent measurements of the physiological characteristic, but also extends to a prediction of the measurements of the physiological characteristic or a prediction of how the physiological characteristic is likely to change in the near future, for example over the next few days following the current measurement of the physiological characteristic. This embodiment allows a warning of an undesirable change in the physiological characteristic to be provided to the subject earlier than in embodiments that just make use of current and recent measurements of the physiological characteristic. Techniques for determining the predictions of the measurements of the physiological characteristic are described in more detail below.

As noted above, the second time interval is shorter than the first time interval. In embodiments where upper and lower global trend lines are determined from maxima and minima in the measurements in the first time interval, it is preferable for those maxima and minima that contribute to the global trend lines to be spaced over a time interval greater than the second time interval. That is, the maxima and minima that contribute to the global trend lines should be further apart than the time covered by the second time interval.

It will be appreciated that steps 105 and 107 can be performed each time that a new physiological characteristic measurement is obtained. Alternatively, step 107 may be performed each time that a new physiological characteristic measurement is obtained, and step 105 may be performed less frequently, for example, once X new physiological characteristic measurements have been obtained (where X>1). In general, step 105 may be performed less frequently than step 107 (i.e. the global trend may be updated less frequently that the local trend).

After the local trend has been determined in step 107, it is determined whether feedback to the subject is required based on the determined global trend, the determined local trend and the desired trend (step 109). Step 109 can be performed or implemented by the determining module 32. If feedback is required, the feedback is provided to the subject in step 111 (for example using user interface component 40). Step 111 can be performed or implemented by the feedback module 34. It will be appreciated that to implement this step the feedback module 34 in the control unit 22 can provide suitable control signals to the user interface component 40 to present the feedback to the subject. The feedback in step 109 can comprise a warning that the physiological characteristic is deviating from the desired trend, and/or one or more coaching messages that provide information or instructions (verbal, textual, or otherwise) to the subject to advise or coach the subject on ways to behave or to change their behaviour and improve the compliance of the physiological characteristic to the desired trend. This feedback is generally referred to as ‘negative’ feedback in the sense that it indicates that the physiological characteristic is deviating from the desired trend.

As noted above, it is desirable to avoid providing a subject with feedback in the form of a warning or coaching message when their physiological characteristic is fluctuating normally, and likewise, when a subject's physiological characteristic is intentionally increasing or decreasing, there will be fluctuations in the physiological characteristic measurements as the physiological characteristic increases or decreases, and it is again desirable to avoid providing a subject with a warning or coaching message while their physiological characteristic is still generally heading in the right direction. However, feedback in the form of a warning or coaching message should be provided to the subject as early as possible. Thus, step 107 achieves this balance by using the determined global trend, the determined local trend and the desired trend to identify situations in which the subject's physiological characteristic is likely to be deviating from the desired trend and in which feedback should be provided to the subject.

Briefly, the invention aims to restrict negative feedback, for example warnings or coaching messages that are intended to encourage the subject to change their behaviour, to times when the deviation is on a global scale. In particular, if the local trend is within the global upper and lower trend lines then generally negative feedback is not provided to the subject in step 109, regardless of whether the local trend is deviating from a previous (e.g. preceding) local trend. However, if the local trend is outside of one of the global trend lines, then (negative) feedback can be provided to the subject. In this way, the natural fluctuations of the physiological characteristic should not lead to warning messages and unnecessary concern for the subject.

It may be possible that the global trend lines indicate a global increase in the physiological characteristic, as with trend lines 15 and 17 in FIG. 4. In that case, it can be that the local trend of the physiological characteristic (as shown by the solid line in FIG. 4) is also increasing while not moving outside the global trend lines (for example as shown from day 20 to day 35 in FIG. 4). To that end, to determine if feedback is required, the value of the global trend lines can be tested to its value some time ago, e.g. a week ago, and in case the difference exceeds a threshold, a warning is issued.

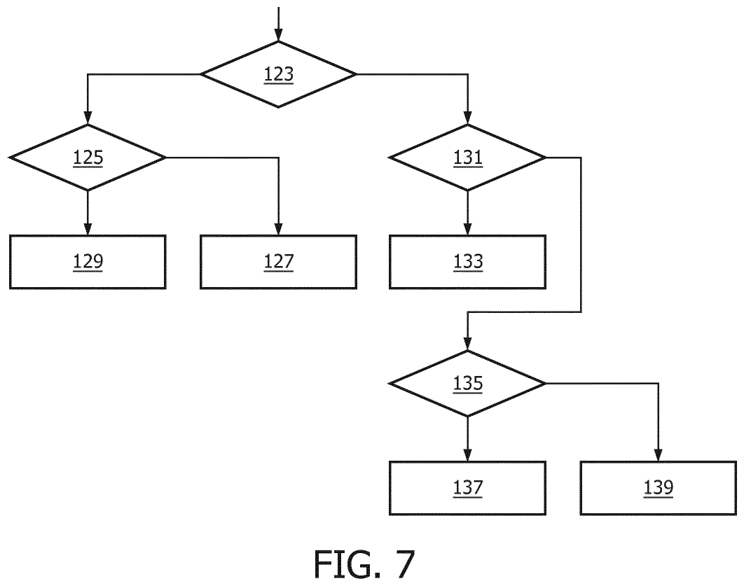

FIG. 7 illustrates an exemplary decision process for implementing step 109 in FIG. 6.

Thus, the global trend (including global upper and lower trend lines), the desired trend and the local trend for the subject are provided as inputs (121) to the process. In a first step (step 123) it is determined whether the global trend is consistent with the desired trend. This step can be implemented by comparing the global trend (e.g. in terms of the global upper and lower trend lines) to the desired trend. For example, consistency between the global trend and the desired trend can be determined by comparing the distance between the desired trend line and the upper and lower global trend lines at certain time points, and if the distance to either global trend line at a particular time point is less than a threshold value, it can be determined that the global trend is not consistent with the desired trend. Otherwise, the global trend can be considered consistent with the desired trend. Optionally, as a check it can be determined if the desired trend is between the upper and lower global trend lines. In a further or alternative embodiment, the gradient of the desired trend can be compared to the gradient of the upper and lower global trend lines, and if the gradients differ by more than a threshold value, the global trend can be considered to be inconsistent with the desired trend.

In the example shown in FIG. 4, the increase in weight from day 20 onwards and bounded by global trend lines 15, 17 may be undesired (i.e. the desired trend could be for the weight to remain generally constant, e.g. as it is up to day 20). In this case, step 123 may identify an inconsistency between the global trend and the desired trend from day 20 onwards.

If the global trend is found to be consistent with the desired trend, then the decision on whether to provide (negative) feedback is reduced to a comparison of the local trend to the global trend and the method moves to step 125. Thus, at step 125 it is determined whether the local trend is within the global trend boundary (i.e. between the global upper trend line and the global lower trend line). This step can be performed by determining if the value of the local trend exceeds the global upper trend line at a particular time point, or if the value of the local trend is below the global lower trend line at a particular time point. If the local trend is not within the global trend boundary the method moves to step 127, and it is decided that feedback in the form of a warning, negative feedback and/or a coaching message should be provided to the subject (step 127). If the local trend is within the global trend boundary at step 125 then the method moves to step 129, and no feedback is required (step 129) and no further action is taken. In the example of FIG. 4 (and assuming that the desired trend is for a weight increase that is generally consistent with the global trend from day 20 onwards), step 127 will identify that the local trend has exceeded the global upper trend line at day 38.

If it is found at step 123 that the global trend is not consistent with the desired trend, then in addition to comparing the local trend to the global upper and lower trend lines, the way in which the local trend may be approaching or crossing a global trend line is important for determining whether feedback is required. For example, exceeding a global upper trend line may be seen to be negative or falling below a global lower trend line may be seen to be positive if the desired trend is for the physiological characteristic to decrease.

Thus if it is found at step 123 that the global trend is not consistent with the desired trend, then the method moves to step 131 where it is determined whether the local trend is consistent with the desired trend (step 131). That is, given that global trend is not consistent with the desired trend (for example the subject may be gaining weight whereas the aim may be for them to lose weight), it is necessary to determine if the local trend is now consistent with the desired trend (e.g. the subject may now have started to lose weight). Step 131 can be performed using similar techniques to those used to perform step 123. If the local trend is consistent with the desired trend at step 131, then the method moves to step 133 and no negative feedback is required for the subject (step 133), and no action is taken.

If the local trend is not consistent with the desired trend (e.g. in the example above the subject has started to maintain their weight or continued the global trend of gaining weight) then, in some embodiments (which are not shown in FIG. 7), feedback in the form or a warning and/or coaching message can be provided to the subject.

In alternative embodiments, if the local trend is not consistent with the desired trend at step 131, then it can be determined at step 135 if the local trend is within the global trend boundaries (similar to step 125). If the local trend is still within the global trend boundaries at step 135, then the warning or coaching message can be suppressed, and no feedback will be provided to the subject (step 137). However, if the local trend is outside the global boundaries at step 135, then negative feedback, coaching messages or a warning can be provided to the subject (step 139).

As noted above, in some embodiments, the second time interval can include upcoming activities and/or events, and thus the local trend can be based on the measurements obtained during the second time interval and a prediction of how upcoming events might affect the physiological characteristic being monitored.

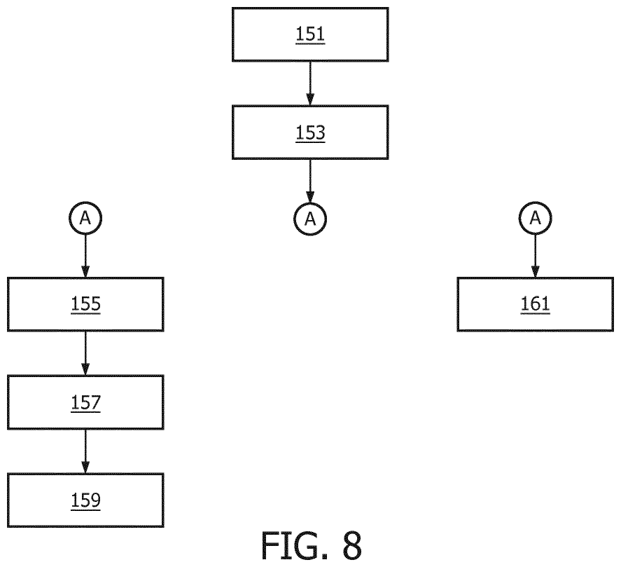

The method in FIG. 8 illustrates an exemplary way in which this can be implemented. The steps in this method can be combined with the steps in the method shown in FIG. 6. In a first step, step 151, information on the activities, events and/or behaviour of the subject are obtained for the period of time in which the measurements of the physiological characteristic are obtained. As with step 101, the information obtained in step 151 can be obtained as the activities, events and/or behaviour occurs, or the information can be retrieved from a memory or other form of data storage.

Next, the information on the activities, events and/or behaviour of the subject and the measurements of the physiological characteristic are analysed to determine associations between certain activities, events and/or behaviour and increases, decreases and stability in the physiological characteristic (step 153). Step 153 is described in more detail below, but for example, an association may be found between the subject being on holiday and weight increasing (due to less activity and eating more), and/or an association may be found between the subject facing a number of work deadlines or presentations and weight decreasing (due to stress affecting appetite).

In order to predict how the physiological characteristic may change in the short-term (i.e. within the second time interval), information on upcoming activities, events and/or behaviour of the subject during the second time interval is obtained (step 155). As described in more detail below, this information can be obtained by examining an electronic schedule or calendar for the subject, and/or the information can be manually provided by the subject or other user of the apparatus 20.

Next, in step 157, the associations determined in step 153 are used to analyse the upcoming activities, events and/or behaviour to determine whether those activities, events and/or behaviour of the subject are likely to increase the physiological characteristic, decrease the physiological characteristic or keep the physiological characteristic stable. In some embodiments, the analysis in step 157 can be restricted to those upcoming activities, events and/or behaviour that are a deviation from the normal routine activities, events and/or behaviour for the subject.

As an example of step 157, if a holiday is scheduled within the time window covered by the second time interval, then step 157 can predict that the subject's weight will increase based on the association found in step 153 above. Step 157 is also described in more detail below.

In step 159, the prediction of whether the upcoming activities, events and/or behaviour are likely to increase the physiological characteristic, decrease the physiological characteristic or keep the physiological characteristic stable is then included as part of the local trend determined in step 107 of FIG. 6.

In a further or alternative embodiment, once the associations between historic activities, events and/or behaviour and increases, decreases and/or stability in the physiological characteristic have been determined, the feedback (particularly the coaching message provided to the subject in step 111) can comprise a recommended activity, event and/or behaviour that is associated with the desired trend for the physiological characteristic. That is, as shown in step 161, the feedback to the subject can recommend one or more activities, events and/or behaviour based on the desired trend and the determined associations. For example, if an association has been found between the subject eating few meals out and weight loss, and it is desired for the subject to lose weight, the feedback provided to the subject in step 111/113 can be to eat less meals out.

As noted above, the analysis in step 157 can be based on upcoming activities, events and/or behaviour that are a deviation from the normal routine activities, events and/or behaviour for the subject. Thus, it is necessary in these embodiments to determine the normal routine for the subject. In some embodiments, the personal routine can be determined according to a personal schedule, which is comprises an occupational (e.g. work) and leisure-time schedule; and/or personal behaviour, which is, e.g., characterized by an eating, activity and sleep pattern.

Data regarding the occupational schedule can be derived from user inputs and connected calendar software tools. The following information may be used to represent the occupational situation/schedule and determine whether from a work perspective the subject has experienced or is about to experience a modification to his/her own habit that influences the physiological characteristic: Home-days/work-days/holidays, Time of year, Working hours and shifts, Occupational tasks and meetings, Number of days until the next deadline, Achievements, Travels, etc.

Similarly, information on the subject's leisure-time schedule can be derived from direct user input, calendar software tools and/or social media. The following information may be used to represent the leisure-time situation and determine whether the subject's habit may change and impact the physiological condition: Social events, Festivities, Appointments, Sports, Sporting events, Shopping schedule, etc.

Additionally behavioural information can be recoded from direct user input or data captured by wearable, mobile, and/or connected environmental sensors. The following aspects can be obtained to determine whether the eating and activity behaviour of the subject has experienced a significant change in a determined time interval: Eating schedule, Eating location, Eating duration, Food diaries, Activity and sedentary time, Sleep, Mental stress level, etc.

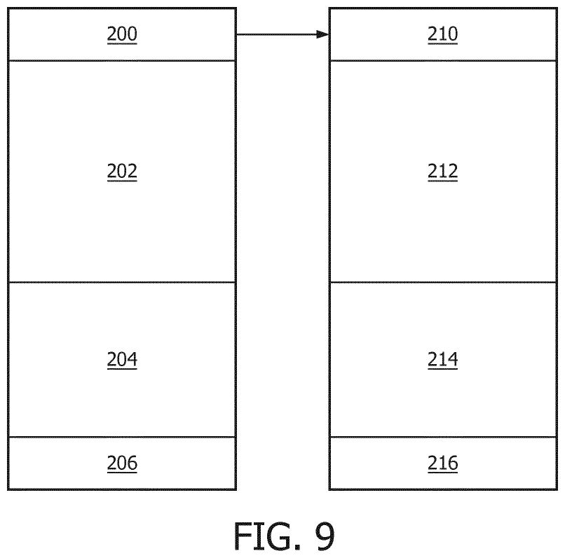

The items listed above represent features that can be used to characterize in a multi-parametric way the habits/routine of a subject. Statistical learning techniques can be used to cluster a subject's situations over time (see FIG. 9) and detect anomalies or transitions between situations. These anomalies and transitions represent the events inducing modifications to the personal routine. FIG. 9 depicts an example of how a clustering of two typical subject situations may be represented by features of the occupational schedule (202; 212), leisure-time schedule (202; 212) and behaviour (204; 214). Each cluster is associated with a certain personal routine (204; 214) to which a specific weight change (increase and stable in FIG. 9) may apply. Any transition from the states represented by the two clusters can be labelled as an event leading to modification to the personal routine, and in the example of FIG. 9 the risk for an increase in weight can be associated with both the transition event as well as to the eating behaviour attained in the (N+1)-cluster.

Thus, in FIG. 9 the (N)-Cluster 200 has features 202 of: office day, 60 days from a work deadline, sunny weather, no social events, no planned appointments and a regular sport schedule. The (N)-Cluster 200 also has the following behaviours 204: 3 meals per day, dinner being the largest meal and the meals being consumed at home. This cluster 200 results in body weight being stable 206. In contrast, the (N+1)-Cluster 210 has features 212 of: office day, 7 days from a work deadline, rainy weather, travelling and an irregular sport schedule. This cluster 210 has the following behaviours 214: 2 meals per day, lunch being the largest meal, eating at a restaurant, and frequent snacking. This cluster 210 results in body weight gain 216.

Statistical and machine learning tools from the field of text and speech recognition are examples of computational methods that can be used to determine situations and clusters from a list of features (occupational, leisure-time and activity) influencing the personal routine. In particular, topic models can be applied to generate clusters of high-level situations. Topic models are used in text and speech recognition to determine associations between words in a sentence and a topic to which they belong. Similarly, in the invention the feature described above could represent primitive characteristics of the situations, which are represented by a discrete number of clusters (topics).

In some embodiments, in view of the normal daily fluctuations in a physiological characteristic, measurements of the physiological characteristic obtained at different times of the day can be corrected or normalised according to the time of day that the measurement was made. This normalisation can help to improve the accuracy of the monitoring provided by the invention by removing some of the measurement fluctuations that occur throughout the day.

In some embodiments, depending on the physiological characteristic being measured, the activity level of the subject in the period preceding the measurement can be taken into account and used to correct measurements of the physiological characteristic. The activity level can be measured with a motion sensor, for example an accelerometer, that is worn by the subject.

In some embodiments, particularly relating to the monitoring of weight, measurements of body hydration by means of, for example, electrical bio-impedance measurements, can be used to better evaluate fluctuations in weight that are not related to fat and muscle mass accumulation or reduction.

In some embodiments, a further refinement of the method could allow for the defining of personalized rules based on historical data from a subject. For example, if a subject has gained a certain amount of weight (e.g. 10%), the past daily measurements could be used to generate subject-specific rules indicating consistent weight change. Indeed, some subjects may experience monotonic increases or decreases in weight, and this information could be used as an alternative to observing trends in relation to local minimum and maximum values. Templates of weight fluctuations on a specific time scale may also be derived to establish whether deviation for such expected weight fluctuations have occurred.

There is therefore provided an improved apparatus and method for monitoring a subject.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer-implemented method of monitoring a subject, the method comprising:
obtaining measurements of a physiological characteristic of the subject over a period of time using a sensor;
obtaining information on a desired trend for the physiological characteristic of the subject;
analysing the measurements of the physiological characteristic in a first time interval to determine a global trend having a global upper trend line for the physiological characteristic based on maxima in the measurements in the first time interval and a global lower trend line for the physiological characteristic based on minima in the measurements in the first time interval;
analysing the measurements of the physiological characteristic in a second time interval to determine a local trend for the physiological characteristic, wherein the second time interval is shorter than the first time interval;
determining whether feedback to the subject is required based on the global trend, the local trend and the desired trend; and
if feedback is required, providing the feedback to the subject via a user interface.

2. A computer-implemented method as claimed in claim 1, wherein the step of analysing the measurements of the physiological characteristic in the first time interval comprises:
determining the global upper trend line as a line that connects two or more maxima in the measurements of the physiological characteristic in the first time interval, and determining the global lower trend line as a line that connects two or more minima in the measurements of the physiological characteristic in the first time interval.

3. A computer-implemented method as claimed in claim 2, wherein the two or more maxima are a greater time apart than the second time interval and the two or more minima are a greater time apart than the second time interval.

4. A computer-implemented method as claimed in claim 1, wherein the step of analysing the measurements of the physiological characteristic in the second time interval to determine the local trend for the physiological characteristic comprises determining the local trend as a moving average of the measurements of the physiological characteristic in the second time interval.

5. A computer-implemented method as claimed in claim 1, wherein the step of determining whether feedback to the subject is required comprises:
determining whether the global trend is consistent with the desired trend; and
determining whether the local trend is between the global upper trend line and the global lower trend line.

6. A computer-implemented method as claimed in claim 1, wherein the step of determining whether feedback to the subject is required comprises:
determining whether the global trend is consistent with the desired trend;
if the global trend is consistent with the desired trend, determining whether the local trend is between the global upper trend line and the global lower trend line;
determining that negative feedback to the subject is required if the local trend is not between the global upper trend line and the global lower trend line;
if the global trend is not consistent with the desired trend, determining whether the local trend is consistent with the desired trend;
determining that negative feedback to the subject is required if the local trend is not consistent with the desired trend; and
otherwise, determining that no feedback to the subject is required.

7. A computer-implemented method as claimed in claim 1, wherein the step of determining whether feedback to the subject is required comprises:
determining whether the global trend is consistent with the desired trend;
if the global trend is consistent with the desired trend, determining whether the local trend is between the global upper trend line and the global lower trend line;
determining that negative feedback to the subject is required if the local trend is not between the global upper trend line and the global lower trend line;
if the global trend is not consistent with the desired trend, determining whether the local trend is consistent with the desired trend;
if the local trend is not consistent with the desired trend, determining whether the local trend is between the global upper trend line and the global lower trend line;
determining that negative feedback to the subject is required if the local trend is not between the global upper trend line and the global lower trend line; and
otherwise, determining that no feedback to the subject is required.

8. A computer-implemented method as claimed in claim 1, wherein the method further comprises the steps of:
obtaining information on the activities, events and/or behaviour of the subject over said period; and
analysing the information on the activities, events and/or behaviour and the measurements of the physiological characteristic to determine associations between certain activities, events and/or behaviour of the subject and increases in the physiological characteristic, decreases in the physiological characteristic and/or stability in the physiological characteristic.

9. A computer-implemented method as claimed in claim 8, wherein if it is determined that feedback is required, the method further comprises the step of:
determining the feedback to be provided to the subject based on the desired trend and the determined associations.

10. A computer-implemented method as claimed in claim 8, wherein the method further comprises the steps of:
obtaining information on upcoming activities, events and/or behaviour for the subject in the second time interval;
using the determined associations to analyse the upcoming activities, events and/or behaviour to predict whether the upcoming activities, events and/or behaviour will lead to an increase in the physiological characteristic, a decrease in the physiological characteristic and/or the physiological characteristic being stable.

11. A computer-implemented method as claimed in claim 10, wherein the step of analysing the measurements of the physiological characteristic in the second time interval to determine the local trend for the physiological characteristic comprises using the measurements of the physiological characteristic in the second time interval and the prediction based on upcoming activities, events and/or behaviour for the subject to determine the local trend.

12. A computer program product comprising a computer readable medium having computer readable code embodied therein, the computer readable code being configured such that, on execution by a suitable computer or processor, the computer or processor is caused to perform the method of claim 1.

13. An apparatus for monitoring a subject, the apparatus comprising a control unit and a user interface, wherein the control unit comprises:
a first obtaining module for obtaining measurements of a physiological characteristic of the subject over a period of time;
a second obtaining module for obtaining information on a desired trend for the physiological characteristic of the subject;
a first analysing module for analysing the measurements of the physiological characteristic in a first time interval to determine a global trend having a global upper trend line for the physiological characteristic based on maxima in the measurements in the first time interval and a global lower trend line for the physiological characteristic based on minima in the measurements in the first time interval;
a second analysing module for analysing the measurements of the physiological characteristic in a second time interval to determine a local trend for the physiological characteristic, wherein the second time interval is shorter than the first time interval;
a determining module for determining whether feedback to the subject is required based on the global trend, the local trend and the desired trend;
and wherein the user interface is configured to provide the feedback to the subject if it is determined that feedback is required.

14. An apparatus as claimed in claim 13, wherein the first analysing module is configured to analyse the measurements of the physiological characteristic in the first time interval by determining the global upper trend line as a line that connects two or more maxima in the measurements of the physiological characteristic in the first time interval, and determining the global lower trend line as a line that connects two or more minima in the measurements of the physiological characteristic in the first time interval.

15. An apparatus as claimed in claim 14, wherein the second analysing module is configured to analyse the measurements of the physiological characteristic in the second time interval to determine the local trend for the physiological characteristic by determining the local trend as a moving average of the measurements of the physiological characteristic in the second time interval.
